(19) United States
(12) Patent Application Publication
(10) Pub. No.: US 2026/0191509 A1
(43) Pub. Date: Jul. 9, 2026

(54) ULTRASOUND IMAGING THREE-DIMENSIONAL (3-D) VISUALIZATION FROM TWO-DIMENSIONAL (2-D) ULTRASOUND IMAGES WITHOUT A 3-D RECONSTRUCTION

(71) Applicant: GE Precision Healthcare LLC, Waukesha, WI (US)

(72) Inventors: Amalie Sofie Ekstrand, Copenhagen N (DK); Anders Beck Olin, Brønshøj (DK); Bo Martins, Rodovre (DK); Mads Andersen, Copenhagen N (DK)

(73) Assignee: GE Precision Healthcare LLC, Waukesha, WI (US)

(21) Appl. No.: 19/014,712

(22) Filed: Jan. 9, 2025

(51) Int. Cl. A61B 8/00; A61B 8/12

(52) U.S. Cl. CPC A61B 8/5207; A61B 8/12; A61B 8/4245; A61B 8/463; A61B 8/466

(57) ABSTRACT

An ultrasound imaging system includes an ultrasound imaging probe with a transducer array configured to emit ultrasound pressure fields and receive echo signals during a sweep of the probe and a tracking sensor configured to track a spatial location and orientation of the ultrasound imaging probe during the sweep, a beamformer configured to generate a set of 2-D ultrasound images for the sweep based on the received echo signals, a visualization module configured to segment tissue of interest in each 2-D ultrasound image of the set of 2-D ultrasound images, generate a mask image of the tissue of interest for each segmented image, convert each mask image to a 3-D graphical representation of the tissue of interest and arrange the 3-D graphical representations in 3-D space based on corresponding tracked spatial location and orientation, and a display monitor configured to present the 3-D graphical representations as a 3-D visualization.

The following generally relates to ultrasound imaging, and finds particular

application to ultrasound imaging three-dimensional (3-D) visualization from two-dimensional (2-D) ultrasound images without a 3-D reconstruction.

### BACKGROUND

Ultrasound imaging provides real-time imaging of information about the interior of a subject such as tissue, organs, etc. An example ultrasound imaging system generally includes an ultrasound imaging probe and a console. The ultrasound imaging probe houses a transducer array, and the console includes or is in electrical communication with a display monitor and a user interface. The transducer array transmits a pressure wave and receives echoes produced in response to the pressure wave interacting with structures such as tissue, blood cells, etc. The echoes are converted to analog signals, which are amplified, digitized, and beamformed to produce scan lines of radio frequency (RF) data. The scan lines are processed (e.g., band-pass filtering, envelope detection, logarithmic compression, etc.), scan converted, and displayed as a two-dimensional (2-D) B-mode ultrasound image.

For three-dimensional (3-D) imaging with a one-dimensional (1-D) transducer array, in one example, the ultrasound imaging probe is swept over a region and a set of 2-D ultrasound images are acquired. A tracking mechanism is utilized to track the ultrasound imaging probe in a 3-D space as the ultrasound imaging probe is swept and 2-D ultrasound images are acquired. An example of such a tracking mechanism is a tracking sensor, which can be integrated in the ultrasound imaging probe and/or attached at an external location on the ultrasound imaging probe. The tracking sensor is configured to track a spatial location and orientation of the transducer array as the ultrasound imaging probe is swept. As such, a spatial location and orientation of each of the 2-D ultrasound images relative to the other 2-D ultrasound images in the 3-D space is tracked.

To generate a 3-D ultrasound image, in one instance, the 2-D ultrasound images are reconstructed using a 3-D reconstruction algorithm. For example, with one algorithm, each of the 2-D ultrasound images is mapped to a corresponding position in a predefined 3-D grid equidistant from each other based on tracked spatial location and orientation information, and interpolation, filtering, and/or other imaging processing techniques are utilized to estimate missing data, e.g., data between the 2-D ultrasound images, and resample the 2-D ultrasound images to generate a 3-D ultrasound image with smooth transitions between the 2-D ultrasound images. To create a 3-D ultrasound image of only tissue of interest, the tissue of interest is first segmented from the 2-D ultrasound images. FIGS. 1, 2, 3 and 4 illustrate an example of a prior art 3-D reconstruction of tissue of interest from a set of 2-D ultrasound images.

Initially referring to FIG. 1, an example acquisition of a set of 2-D ultrasound images is schematically illustrated. In this example, an ultrasound imaging probe 102 is swept 104 over a surface 106 of a subject 108 at a location 110 above tissue of interest 112 inside of the subject 108, and a transducer array 114 of the ultrasound imaging probe 102 acquires a set of 2-D ultrasound images of the tissue of interest 112 during the sweep. In this example, the tissue of interest 112 is part of a blood vessel tree. Moving to FIG. 2, a tissue of interest mask 202 for one of the 2-D ultrasound images is schematically illustrated. For this, the tissue of interest is segmented in the 2-D ultrasound image to create a segmented 2-D ultrasound image. Then, the tissue of interest mask 202 is generated based on the segmented 2-D ultrasound image by masking out all tissue except segmented tissue of interest 204. This is performed for the set of 2-D ultrasound images.

Next at FIG. 3, part of an example 3-D reconstruction is schematically illustrated. In this example, for explanatory purposes, the 2-D ultrasound images (and not the tissue of interest masks generated therewith) are shown positioned in a 3-D grid 302 based on tracked spatial location and orientation information for each 2-D ultrasound image, where image processing (e.g., interpolation, filtering, etc.) is performed to reconstruct a 3-D volume 304 from the set of 2-D ultrasound images in the 3-D grid 302. Only a single x, y plane, a single x, z plane and a single y, z plane of the reconstructed 3-D ultrasound volume are shown in FIG. 3 for clarity purposes, but it is to be understood that a 3-D volume is generated. FIG. 4 schematically illustrates a 3-D ultrasound image 402 of the tissue of interest generated with the 3-D reconstruction. In this example, the 3-D ultrasound image includes only a 3-D representation of the tissue of interest 112 (FIG. 1), i.e., the part of a blood vessel tree.

In general, 2-D ultrasound images are first acquired by sweeping an ultrasound imaging probe over a region to acquire a set of 2-D ultrasound images and then the set of 2-D ultrasound images are processed (e.g., segmented, converted to masks, positioned in a 3-D grid, resampled, filtered, etc.) with a 3-D reconstruction algorithm to generate a single 3-D ultrasound volume. With this process, there is a latency between the acquisition of the set of 2-D ultrasound images and the generation of the 3-D ultrasound volume, and, as a consequence, the 3-D ultrasound volume is displayed only after the acquisition of the set of 2-D ultrasound image. Furthermore, the 3-D reconstruction consumes time and adds computational strain, requiring additional memory and processing resources. Moreover, the 3-D reconstruction algorithm may introduce errors (e.g., stitching, rounding-off, smoothing due to interpolation, etc. between 2-D ultrasound images) and/or may inadvertently hide errors/weaknesses in one or more of the 2-D ultrasound images (e.g., where sampling is uneven, etc.).

In view of at least the foregoing, there is an unresolved need for an improved approach for generating a 3-D ultrasound volume from 2-D ultrasound images.

### SUMMARY

Aspects of the application address the above matters, and others. This summary introduces concepts that are described in more detail in the detailed description. It should not be used to identify essential features of the claimed subject matter, nor to limit the scope of the claimed subject matter.

In one aspect, a system includes an ultrasound imaging system. The ultrasound imaging system includes an ultrasound imaging probe. The ultrasound imaging probe includes a transducer array configured to emit ultrasound pressure fields and receive echo signals during a sweep of the probe. The ultrasound imaging probe further includes a tracking sensor configured to track a spatial location and orientation of the ultrasound imaging probe during the sweep. The ultrasound imaging system further includes a beamformer configured to generate a set of 2-D ultrasound images for the sweep based on the received echo signals. The ultrasound imaging system further includes a visualization module configured to segment tissue of interest in each 2-D ultrasound image of the set of 2-D ultrasound images. The visualization module is further configured to generate a mask image of the tissue of interest for each segmented image of the set of 2-D ultrasound images. The visualization module is further configured to convert each mask image to a 3-D graphical representation of the tissue of interest. The visualization module is further configured to arrange the 3-D graphical representations in 3-D space based on corresponding tracked spatial location and orientation. The ultrasound imaging system further includes a display monitor configured to present the 3-D graphical representations as a 3-D visualization.

In one or more instances, the 3-D visualization includes gaps between neighboring 3-D graphical representations. In one or more instances, additionally or alternatively, the 3-D graphical representation is a 3-D point cloud. In one or more instances, additionally or alternatively, the display monitor displays the 3-D visualization after arrangement of a 3-D graphical representation in the 3-D space. In one or more instances, additionally or alternatively, the display monitor updates the display of the 3-D visualization after an addition of a subsequent 3-D graphical representation in the 3-D space. In one or more instances, additionally or alternatively, the visualization module is configured to segment a perimeter of each 2-D ultrasound image, include the segmented perimeters in the mask image, include the segmented perimeters with the 3-D graphical representation, and display the segmented perimeters in the 3-D visualization. In one or more instances, additionally or alternatively, the sweep of the ultrasound imaging probe is a free-hand sweep of the ultrasound imaging probe. In one or more instances, additionally or alternatively, the ultrasound imaging probe is a laparoscopic ultrasound imaging probe.

In another aspect, a method includes receiving echo signals with a transducer array of an ultrasound imaging probe during a sweep of the probe. The method further includes tracking a spatial location and orientation of the ultrasound imaging probe during the sweep. The method further includes generating a set of 2-D ultrasound images for the sweep based on the received echo signals. The method further includes segmenting tissue of interest in each 2-D ultrasound image of the set of 2-D ultrasound images. The method further includes generating a mask image of the tissue of interest for each segmented image of the set of 2-D ultrasound images. The method further includes converting each mask image to a 3-D graphical representation of the tissue of interest. The method further includes arranging the 3-D graphical representations in 3-D space based on corresponding tracked spatial location and orientation. The method further includes displaying the 3-D graphical representations as a 3-D visualization.

In another aspect, a computer readable medium encoded with computer executable instructions, which, when executed by a processor, cause the processor to receive echo signals with a transducer array of an ultrasound imaging probe during a sweep of the probe. The computer executable instructions further causes the processor to track a spatial orientation of the ultrasound imaging probe during the sweep. The computer executable instructions further causes the processor to generate a set of 2-D ultrasound images for the sweep based on the received echo signals. The computer executable instructions further causes the processor to segment tissue of interest in each 2-D ultrasound image of the set of 2-D ultrasound images. The computer executable instructions further causes the processor to generate a mask image of the tissue of interest for each segmented image of the set of 2-D ultrasound images. The computer executable instructions further causes the processor to convert each mask image to a 3-D graphical representation of the tissue of interest. The computer executable instructions further causes the processor to arrange the 3-D graphical representations in 3-D space based on corresponding tracked spatial location and orientation. The computer executable instructions further causes the processor to display the 3-D graphical representations as a 3-D visualization.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates sweeping an ultrasound imaging probe to acquire a set of acquired 2-D images of tissue of interest.

FIG. 2 schematically illustrates a tissue of interest map generated via segmenting tissue of interest in one of the 2-D images of the set of acquired 2-D images.

FIG. 3 schematically illustrates part of a 3-D volume generated with a 3-D reconstruction using the set of acquired 2-D images of tissue of interest.

FIG. 4 schematically illustrates a reconstructed 3-D ultrasound image of the tissue of interest.

FIG. 5 schematically illustrates a non-limiting example of a imaging system configured for ultrasound imaging and including a 3-D visualization module, in accordance with an aspect of an embodiment(s) herein.

FIG. 6 schematically illustrates a non-limiting example of the 3-D visualization module, in accordance with an aspect of an embodiment(s) herein.

FIG. 7 graphically illustrates a side view of acquiring 2-D ultrasound images of tissue of interest by free-hand sweeping of an ultrasound imaging probe over a region of a subject, in accordance with an aspect of an embodiment(s) herein.

FIG. 8 graphically illustrates a perspective view of acquiring the 2-D ultrasound images of tissue of interest with the free-hand sweep of the ultrasound imaging probe at a particular point, in accordance with an aspect of an embodiment(s) herein.

FIG. 9 graphically illustrates a mask image for the tissue of interest in one of the 2-D ultrasound images, in accordance with an aspect of an embodiment(s) herein.

FIG. 10 graphically illustrates a side view of building up of a 3-D visualization from 3-D point clouds generated from acquired 2-D ultrasound images, in accordance with an aspect of an embodiment(s) herein.

FIG. 11 graphically illustrates a perspective view of building up of the 3-D visualization from 3-D point clouds generated from acquired 2-D ultrasound images, in accordance with an aspect of an embodiment(s) herein.

FIG. 12 graphically illustrates an example of a 3-D visualization built up from 3-D point clouds corresponding to the acquired 2-D ultrasound images, in accordance with an aspect of an embodiment(s) herein.

FIG. 13 illustrates a non-limiting example of a flow chart for a method of creating a 3-D visualization from 2-D ultrasound images acquired with a free hand sweep of the ultrasound imaging probe and without a 3-D reconstruction, in accordance with an embodiment(s) herein.

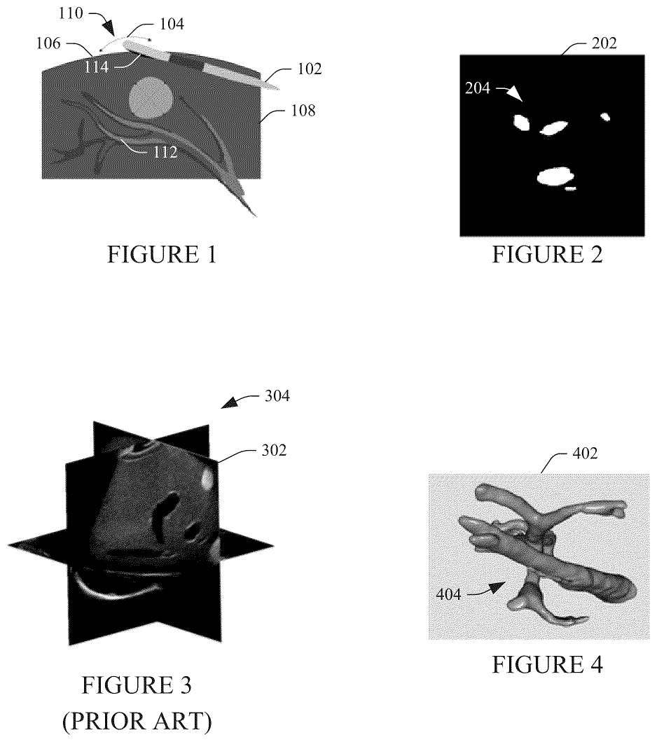

### DETAILED DESCRIPTION

Embodiments of the present disclosure will now be described, by way of example, with reference to the figures, in which a system, a method and/or instructions encoded in a computer readable medium generate an ultrasound three-dimensional (3-D) visualization based on 2-D ultrasound images and their spatial location and orientation with respect to each other in 3-D space, without a 3-D reconstruction. As utilized herein, the terms reconstructing, reconstruction, and the like involve creating data between the 2-D ultrasound images and/or modifying parts of the 2-D ultrasound images to combine the 2-D ultrasound images together and create a single 3-D ultrasound image.

As discussed above, such a 3-D reconstruction adds latency between acquisition and generation of the single 3-D ultrasound image, e.g., at least because the 2-D ultrasound images are acquired and arranged in the 3-D grid before the 3-D reconstruction is performed, and the 3-D reconstruction itself consumes time. Furthermore, the 3-D reconstruction adds computational strain on the processing resources, requiring additional memory and processing cycles relative to generating the 2-D ultrasound images. Moreover, the processing to combine the 2-D ultrasound images may introduce errors and/or may inadvertently hide errors/weaknesses from the underlying data.

With the approach described herein, 2-D ultrasound images are acquired by sweeping a transducer array of an ultrasound imaging probe across an area above tissue of interest while tracking the position and/or orientation of the ultrasound imaging probe. As a 2-D ultrasound image that includes the tissue of interest is acquired, the tissue of interest is segmented and the 2-D ultrasound image is converted into a 3-D representation of the tissue of interest. The 3-D representation of the tissue of interest is then arranged in 3-D space based on the tracked position and/or orientation and displayed. This is repeated for each newly acquired 2-D ultrasound image, with the displayed visualization being updated to add the latest 3-D representation of the tissue of interest to the displayed visualization, building up a 3-D visualization.

Since there is no 3-D reconstruction, the approach described herein mitigates the latency, time consumption, computational strain, etc. associated with 3-D reconstructions of 3-D ultrasound images from 2-D ultrasound images. In addition, the displayed 3-D visualization can be constructed in near real-time, taking into consideration time for the segmentation, conversion to and arrangement of the 3-D representations. Moreover, since the individual 3-D representations of the tissue of interest make up the displayed 3-D visualization, shortcomings associated with combining 2-D ultrasound images to create a single 3-D ultrasound image (e.g., introducing errors and/or inadvertently hiding errors/weaknesses) are mitigated.

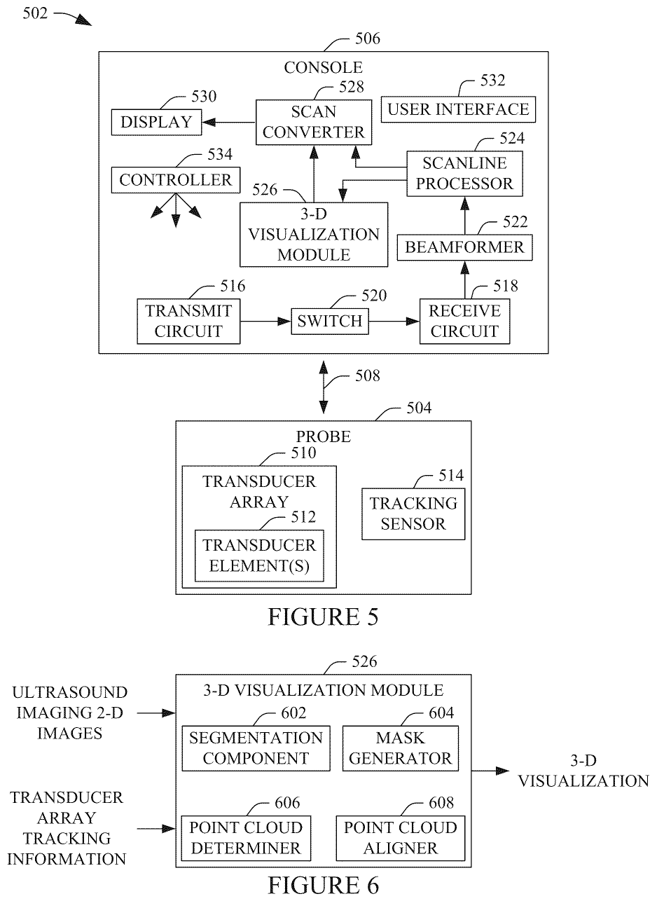

Turning to FIG. 5, a non-limiting example of an imaging system 502 configured for ultrasound imaging (e.g., an ultrasound imaging system) is schematically illustrated. The ultrasound imaging system 502 includes an ultrasound imaging probe 504 and a console 506. The ultrasound imaging probe 504 and the console 506 communicate and/or interface with each other over a communication channel 508. In one instance, the communication channel 508 includes wired technology, e.g., complimentary interfaces and a cable therebetween. In another instance the communication channel 508 includes wireless technology, e.g., Wi-Fi®, Bluetooth®, etc. In yet another instance, the ultrasound imaging probe 504 and the console 506 are integrated in a same housing such as part of a hand-held ultrasound system, etc.

The ultrasound imaging probe 504 includes a transducer array 510. The transducer array 510 includes one or more transducer elements 512. Examples of such arrays include 64, 128, 192, 256, and/or other number of elements, including larger and smaller arrays, one dimensional (1-D) or two dimensional (2-D), etc. The transducer array 510 can be linear, curved, and/or otherwise shaped, fully populated, sparse and/or a combination thereof, etc. In one instance, the ultrasound imaging probe 504 includes a laparoscopic ultrasound imaging probe. In another instance, the ultrasound imaging probe 504 includes another type of ultrasound imaging probe.

The one or more transducer elements 512 are configured to convert an excitation electrical signal to an ultrasound pressure field and convert a reflected ultrasound pressure field to an electrical signal. By way of non-limiting example, the one or more transducer elements 512 can be selectively excited via an excitation electrical (pulsed) signal, which causes at least a sub-set of the transducer elements to transmit an ultrasound pressure field into an examination or scan field of view. The ultrasound pressure field may include a focused ultrasound beam, a defocused (spherical) wave, and/or other ultrasound signal. The one or more transducer elements 512 receive echo signals and generate analog electrical signals indicative thereof. The echo signals are generated in response to the transmitted ultrasound pressure field interacting with structure, such as tissue, blood cells flowing in a portion of a vessel, etc.

As described in greater detail below, for generating a 3-D visualization of tissue of interest in a subject, a plurality of 2-D ultrasound images are acquired and processed. In one instance, the transducer array 510 includes a 1-D array, and the plurality of 2-D ultrasound images are obtained by a free hand sweep of the transducer array 510 over an area above the tissue of interest, where individual 2-D ultrasound images are obtained slice-by-slice. In another instance, the ultrasound imaging probe 504 further includes electro-mechanical components that are configured to rotate the transducer array 510 within the ultrasound imaging probe 504 acquired by the individual 2-D ultrasound images slice-by-slice. In yet another instance, the transducer array 510 includes a 2-D array or matrix, and the plurality of 2-D ultrasound images can be acquired concurrently, e.g., plane-by-plane.

The ultrasound imaging probe 504 includes at least one tracking sensor 514. The at least one tracking sensor 514 is configured to track a spatial orientation and location of the transducer array 510. In one instance, the at least one tracking sensor 514 is integrated in and part of the ultrasound imaging probe 504. In another instance, the at least one tracking sensor 514 is affixed to the ultrasound imaging probe 504 and/or a device affixed to the ultrasound imaging probe 504 and configured to carry the at least one tracking sensor 514. Such a tracking sensor can be located anywhere in and/or on the ultrasound imaging probe 504 where it can track sweeping, rotation, translation, etc. of the transducer array 510. Examples of suitable sensors include single and/or multi-axis electromagnetic, inertial (e.g., a gyroscope, an accelerometer, etc.), optical, etc., sensors.

The console 506 includes a transmit circuit 516 and a receive circuit 518. The transmit circuit 516 is configured to generate the excitation electrical signal provided to the transducer array 510 for transmitting the ultrasound pressure field. In one instance, this includes generating delays to individual elements of the one or more transducer elements 512 for transmit focusing, beam steering, etc. The receive circuit 518 is configured to receive the analog electrical signals from the at least one element and pre-process the analog electrical signals, e.g., amplify, digitize, focus, and/or otherwise process the analog electrical signals.

For example, in one instance the receive circuit 518 includes an amplifier and a corresponding analog to digital converter (ADC) for each element, where each amplifier amplifies a corresponding analog electrical signal from a micro-volt level to a voltage range of the ADC. The console 506 further includes a switch (“SW”) 520 configured to switch between the transmit circuit 516 and the receive circuit 518, e.g., by electrically connecting the transmit circuit 516 to the transducer array 510 for a transmit operation and electrically connecting the receive circuit 518 to the transducer array 510 for a receive operation. In an alternative instance, separate switches are employed for the transmit circuit 516 and the receive circuit 518.

The console 506 further includes a beamformer 522. The beamformer 522 is configured to beamform, e.g., via delay-and-sum (e.g., a matched-filter beamformer, etc.) and/or other beamforming, the signals from the receive circuit 518 and generate radiofrequency (RF) data. With delay-and-sum beamforming, the digital signal for each element is delayed to align the signals in time, amplified, and then summed. In one instance, a matched filter matched to an expected received echo-pulse shape (bandwidth) operates on the signals..

The console 506 further includes a scanline processor 524. When configured for I/Q demodulation, the scanline processor 524 down mixes the RF signal and optionally applies low pass filtering and/or decimation. This may include employing a Hilbert Transform, a combination of a Complex-Demodulation Band Pass Filter and optional decimation, and/or other processing. The scanline processor 524 detects, extracts and outputs an envelope (i.e., an amplitude) of the I/Q signal (or the RF signal where I/Q modulation is omitted). In one instance, this is achieved using a Hilbert Transform and/or other approach.

The scanline processor 524 compresses the extracted envelope, reducing the dynamic range, e.g., to reduce the dynamic range to a predetermined display precision by a logarithmic (log)-based dynamic range compression and/or otherwise, and outputs a scanline. The scanline processor 524 outputs the processed scanlines as a frame/image (e.g., a B-mode image). The scanline processor 524 may apply other processing such as filtering (e.g., via a Finite Impulse Response (FIR) filter, an Infinite Impulse Response (IIR) filter, etc.), Time Gain Compensation (TGC), noise rejection, and/or other processing. Other processing, such as flow, etc. is contemplated herein.

The console 506 further includes a 3-D visualization module 526. The 3-D visualization module 526 is configured to generate an ultrasound three-dimensional (3-D) visualization based on the acquired 2-D ultrasound images and the spatial location and orientation of the acquired 2-D ultrasound images tracked via the tracking sensor 514, without a 3-D reconstruction. Again, a 3-D reconstruction adds latency between acquisition and generation of the single 3-D ultrasound volume, consumes time and increases processing resource requirements (e.g., relative to the generating just the 2-D ultrasound images), and may introduce errors and/or obscure errors in the underlying 2-D ultrasound images.

As described in greater detail below, the approach herein constructs the 3-D visualization with the 2-D ultrasound images, without any 3-D reconstruction, mitigating the shortcomings associated with 3-D reconstructions (e.g., latency, time consumption, increased computational resources, introduction of errors, inadvertent obscuring error in the 2-D ultrasound images, etc.). In addition, the displayed 3-D visualization can be constructed in near real-time (e.g., as 2-D ultrasound images are acquired and/or become available for processing), providing real-time feedback to the sonographer, who can adjust the ultrasound imaging probe 504 position, adjust imaging parameters, end the scan where it appears a diagnostic quality 3-D visualization may not be possible, etc., based on the real-time feedback.

The console 506 further includes a scan converter 528. The scan converter 528 is configured to scan convert the 2-D ultrasound images and/or the 3-D visualization into a coordinate system of a display 530. The scan converter 528 can be configured to employ analog and/or digital scan converting techniques. In the illustrated example, the display 530 is integrated with the console 506. In another instance, the display 530 is a separate and/or remote display monitor in electrical communication with the console 506.

The console 506 further includes a user interface 532. The user interface 532 includes one or more input devices (such as a button, a knob, a slider, a touch screen, a mouse, a keyboard, etc.) and/or other input device, and/or one or more output devices such as a visible, audible, etc. indicator. The user interface 532 allows a user to control an operation of the ultrasound imaging system 502. The user interface 532 is shown integrated with the console 506. In another instance, the user interface 532 is a separate and/or remote keyboard, keypad, touch screen, etc. in electrical communication/n with the console 506.

The console 506 further includes a controller 534. The controller 534 includes a processor(s) such as a microprocessor (mP), a central processing unit (CPU), a graphics processing unit (GPU), etc., and computer readable storage medium. The computer readable storage medium includes computer readable instructions, and the processor is configured to execute instructions stored in the computer readable storage medium. The controller 534 is configured to control one or more of the transmit circuit 516, the receive circuit 518, the switch 520, the beamformer 522, the scanline processor 524, the 3-D visualization module 526, the scan converter 528, the display 530, and the user interface 532. One or more of the components of the console 506 can be implemented in software and/or hardware.

FIG. 6 schematically illustrates a non-limiting example of the 3-D visualization module 526. The 3-D visualization module 526 includes a segmentation component 602, a mask generator 604, a point cloud determiner 606, and a point cloud aligner 608. The 3-D visualization module 526 receives and/or retrieves, as input, the 2-D ultrasound images generated by the scanline processor 524 (FIG. 5) and ultrasound imaging probe tracking information generated by the tracking sensor 514 (FIG. 5). The 3-D visualization module 526 outputs a 3-D visualization of tissue of interest as a series of separate 3-D graphical representations of the tissue of interest from individual 2-D ultrasound images aligned in 3-D space based on the ultrasound imaging probe tracking location and/or orientation information.

The segmentation component 602 segments the tissue of interest from each 2-D ultrasound image, as the 2-D ultrasound images become available. The segmentation component 602 employs known and/or other segmentation algorithms. Example algorithms include thresholding, clustering, artificial intelligence (AI) such as deep learning models (e.g., a Region-based Convolutional Neural Network (R-CNN) etc.), etc. In one instance, the segmentation is performed automatically by the segmentation component 602, i.e., without user input for the segmentation such as identifying a perimeter of the tissue of interest, etc. In another instance, the segmentation is performed semi-automatically with user input for at least a portion of the segmentation. In one instance, the particular or type of tissue of interest it identified by the user, e.g., via an input, a selection from a menu, etc. In another instance, the segmentation component 602 determines the tissue of interest based on a selected scan protocol.

The mask generator 604 generates a mask for each segmented 2-D ultrasound image. The mask generator 604 employs known and/or other segmentation algorithms. In one instance, the mask generator 604 initializes a blank mask (i.e., all (0's) zeros, etc.) with the same dimensions as the original image, and then fills in the segmented region with a specific value (e.g., all (1's) ones) to create the mask. In another instance, the mask generator 604 begins with the 2-D ultrasound image or a copy of the 2-D ultrasound image and changes values outside of the segmented region with a specific value (e.g., all (0's) zeros, etc.) to create the mask. Other segmentation approaches are also contemplated herein. A mask for a particular segmented 2-D ultrasound image includes one or more regions corresponding to the tissue of interest. In some instances, a mask may include more than one type or different types of tissue of interest. In some instances, the mask may further include an outline representing a perimeter of the original of the 2-D ultrasound image.

The point cloud determiner 606 converts each mask into one or more 3-D collections of data points for the one or more types of the tissues of interest. In one instance, a 3-D collection of data points include a 3-D point cloud, i.e., a sphere with coordinates (x, y, z) that are based on the tracked spatial location and orientation of the ultrasound imaging probe 504. The point cloud determiner 606 employs known and/or other point cloud creation algorithms. In one non-limiting instance, the point cloud determiner 606 loads the mask image, identifies the segmented region(s), extracts the coordinates of the pixels that belong to the segmented region(s), assigns depth information to the 2-D segmented region(s) to create a 3-D representation, and combine the coordinates and depth information to form a 3-D point cloud. In one instance, the data points represent an external surface of the tissue of interest in the corresponding 2-D ultrasound image. In another instance, each point additionally includes additional information such as attributes like color or intensity.

The point cloud aligner 608 aligns each 3-D point cloud in 3-D space based on the tracked spatial location and orientation of the ultrasound imaging probe 504 to create the 3-D visualization. For example, a 3-D point cloud for a 2-D ultrasound image will be placed in the 3-D visualization at a same location as the 2-D ultrasound image is at in the volume covered by the acquired 2-D ultrasound images. As such, the 3-D visualization will include a 3-D point cloud for each 2-D ultrasound image, with each 3-D point cloud arranged in the 3-D space based on the spatial location and orientation of the corresponding 2-D ultrasound image. The 3-D visualization will include gaps without data between each of the 3-D point clouds. The gaps correspond to the spacing between the 2-D ultrasound image and includes no gaps where neighboring 2-D ultrasound images overlap. Hence, the integrity of the acquired 2-D ultrasound image is maintained and data is not derived to create data between 3-D point clouds, nor are the 3-D point clouds manipulated to create smooth transitions between the 3-D point clouds.

The 3-D visualization module 526 outputs the 3-D visualization. As described herein, the 3-D visualization is displayed via the display 530. The 2-D ultrasound image can be concurrently and/or alternately displayed. As described herein, in one instance the 3-D visualization can be displayed and built up as 2-D ultrasound images become available to the 3-D visualization module 526. In another instance, two or more (including all) of the 2-D ultrasound images can be converted into 3-D point clouds and arranged in the 3-D space before the 3-D visualization is displayed via the display 530. The 3-D visualization can be manipulated by known and/or other processing techniques, such as rotated, panned, annotated, warped, magnified, smoothed, sharpened, brightened, etc. Where the mask includes an outline representing a perimeter of each of the original of the 2-D ultrasound images, the 3-D visualization may also include a perimeter of the volume of the 2-D ultrasound images, which may provide context such as the position of the ultrasound imaging probe 504, the direction of the sweep of the ultrasound imaging probe 504, etc.

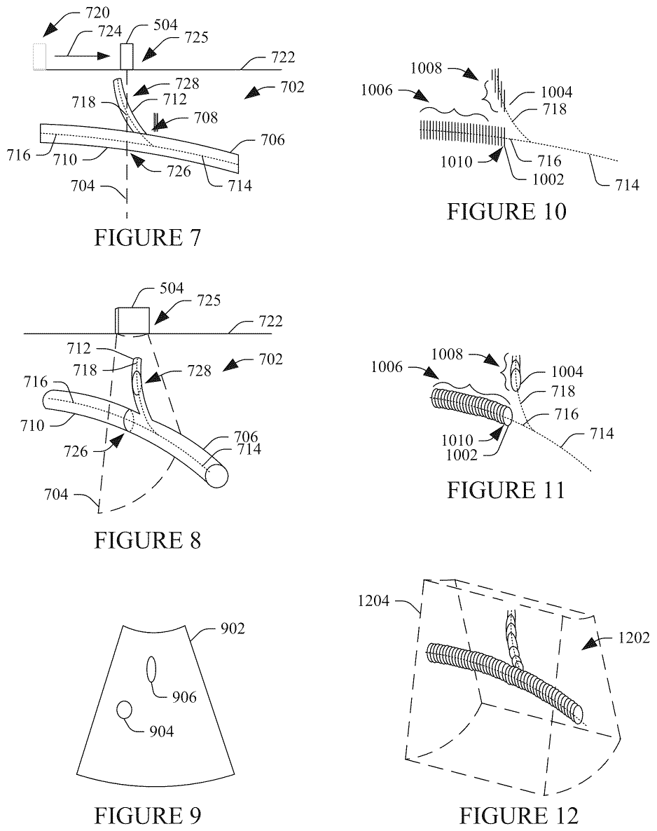

FIGS. 7, 8, 9, 10, 11 and 12 graphically illustrate an example of generating a 3-D visualization using the approach described herein. FIG. 7 graphically illustrates an example of a side view of tissue of interest in connection with the ultrasound imaging probe 504 and an image plane. FIG. 8 graphically illustrates an example of a perspective view of the tissue of interest in connection with the ultrasound imaging probe 504 and the image plane described in connection with FIG. 7. FIG. 9 graphically illustrates an example mask image generated for the image plane described in connection with FIGS. 7 and 8. FIG. 10 graphically illustrates an example of a side view of 3-D point clouds aligned in 3-D space for 2-D ultrasound images up to the mask described in connection with FIG. 9. FIG. 11 graphically illustrates an example of a perspective view of the aligned 3-D point clouds described in connection with FIG. 10. FIG. 12 graphically illustrates an example of a displayed 3-D visualization of the entire scanned tissue of interest.

Initially referring to FIG. 7, an example of a side view of tissue of interest 702 in connection with the ultrasound imaging probe 504 and an image plane 704 is graphically illustrated. In this example, the tissue of interest 702 is tubular tissue of interest such as a blood vessel and/or other tubular tissue of interest. In this example, the tubular tissue of interest 702 includes a main channel 706 and bifurcation 708 at which the main channel 706 divides into multipole branches, including a branch 710, . . . , and a branch 712. The main channel 706 includes a long axis 714, which is illustrated along a centerline of the main channel 706. The branch 708 includes a long axis 716, which is illustrated along a centerline of the branch 708. The branch 710 includes a long axis 718, which is illustrated along a centerline of the branch 710.

In the illustrated example, the ultrasound imaging probe 504 was activated to begin scanning at an initial position 720 on a surface 722 of a subject over the tissue of interest 702. For the scan, the ultrasound imaging probe 504 is swept, via a free-hand sweep, in a direction 724, which is along the long axes 714, 716 and 718 respectively of the main channel 706, the branch 710 and the branch 712. In this example, the ultrasound imaging probe 504 is at a current position 725 of the surface 722 of the subject. The image plane 704 is transverse to the long axes 714, 716 and 718 of the main channel 706, the branch 710 and the branch 712. The image plane 704 extends from the ultrasound imaging probe 504 at the current position 726 and traverses the branch 710 and the branch 712.

At the current position 725, the image plane 704 extends through a cross-section 726 of the branch 710 and a cross-section 728 of the branch 712. Depending on the position of the ultrasound imaging probe 504, in other instances image plane 704 extends only through the branch 710, only through the main channel 706, etc. The number of 2-D ultrasound images acquired from the initial position 720 to the current position 726 and from the current position 725 to the end of the scan and spacing between the 2-D ultrasound images depends on various factors such as frame rate, speed of the free-hand sweep, path of the free-hand sweep, elevation resolution/beam height, etc. It is to be understood that the shape, size, location, etc. of the objects illustrated in FIG. 7 (and FIGS. 8-12) are for explanatory purpose and are not limiting.

With further reference to FIG. 8, a perspective view of the tissue of interest 702, the ultrasound imaging probe 504 (at the current position 725), and the image plane 704 (at the current position 726) described in connection with FIG. 7 is graphically illustrated. In this example, the image plane 704 is fan or sector shaped. In other instance, the image plane can be otherwise shaped, e.g., square, rectangular, etc. In this example, the cross-section 726 of the branch 710 includes a larger disc shaped region relative to the cross-section 728 of the branch 712, which includes a smaller disc shaped region. The remainder of the image plane 704 is illustrated as not traversing any object for clarity and explanatory purposes, however, it is to be understood that the image plane 704 may traverse other tissue, blood cells, etc. with the object.

Moving to FIG. 9, an example mask image 902 generated for the image plane 704 described in connection with FIGS. 7 and 8 is graphically illustrated. In general, the echo signals received by the ultrasound imaging probe 504 are processed to generate a corresponding 2-D ultrasound image, which will include the objects traversed by the image plane 704 at the current location 725. The segmentation component 602 segments the tissue of interest (the cross-section 726 of the branch 710 and the cross-section 728 of the branch 712) as described in connection with FIG. 6 and/or otherwise. For example, the segmentation component 602 can employ thresholding, clustering, a deep learning model, etc. via automated and/or semi-automated approaches to segment the cross-section 726 and the cross-section 728 from the 2-D ultrasound image. The segmentation component 602 can then generate the mask image 902, which only includes a representation 904 of the segmented cross-section 726 of the branch 710 and a representation 906 of the segmented cross-section 728 of the branch 712.

Next at FIG. 10, an example of a side view of 3-D point clouds aligned in 3-D space for 2-D ultrasound images acquired up to the mask image 902 at the current location 726 (FIGS. 7 and 8) and based on the image masks (FIG. 9) is graphically illustrated. In this example, the long axes 714, 716 and 718 respectively of the main channel 706, the branch 710 and the branch 712 (FIG. 7) are provided for a frame of reference and explanatory purposes. The point cloud determiner 606 converts each mask image 902 into one or more 3-D collections of data points for the one or more type of the tissue of interest, as described in connection with FIG. 6 and/or otherwise. The point cloud aligner 608 aligns each 3-D point cloud in 3-D space based on the tracked spatial location and orientation of the ultrasound imaging probe 504 to create the 3-D visualization as described in connection with FIG. 6 and/or otherwise.

In this example, a 3-D point cloud 1002 corresponds to the representation 904 (FIG. 9) of the cross-section 726 of the branch 710 (FIGS. 7 and 8). In addition a 3-D point cloud 1004 corresponds to the representation 906 (FIG. 9) of the cross-section 728 of the branch 712 (FIGS. 7 and 8). 3-D point clouds 1006 correspond to representations of cross-sections of the branch 710 acquired from the initial position 720 to the current position 726 (FIGS. 7 and 8). 3-D point clouds 1008 correspond to representations of cross-sections of the branch 712 acquired from the initial position 720 to the current position 726 (FIGS. 7 and 8).

As discussed herein, spacings between the 2-D ultrasound images depend on various factors such as frame rate, speed of the free-hand sweep, path of the free-hand sweep, elevation resolution/beam height, etc. The spacings between the 2-D ultrasound images will exist between the 3-D point clouds. For example, a spacing 1010 between the 3-D point cloud 1002 and the preceding 3-D point cloud corresponds to a spacing between the 2-D ultrasound image for the 3-D point cloud 1002 and the 2-D ultrasound image for the preceding 3-D point cloud. In contrast, a prior art 3-D reconstruction will resample the 2-D ultrasound images and estimate data for gaps between the 2-D ultrasound images to reconstruct a single 3-D volume with no gaps, even though there were gaps between the original 2-D ultrasound images. The resampling and/or data estimation increases memory and processing requirements. The approach described herein mitigates the increase in memory and processing requirements for such a 3-D reconstruction, e.g., as no 3-D reconstruction, resampling, estimating data to fill gaps between the original 2-D ultrasound images, etc. is performed.

With further reference to FIG. 11, a perspective view of the 3-D point clouds aligned in 3-D space for 2-D ultrasound images acquired up to the mask image 902 at the current location 726 (FIGS. 7 and 8) and based on the image masks (FIG. 9) is graphically illustrated. Again, the long axes 714, 716 and 718 respectively of the main channel 706, the branch 710 and the branch 712 (FIG. 7) are provided for a frame of reference and explanatory purposes.

Turning to FIG. 12 an example of a displayed 3-D visualization 1202 of the entire scanned tissue of interest 702 is graphically illustrated. The example includes the 3-D point clouds described in connection with FIGS. 10 and 11 and 3-D point clouds generated for 2-D ultrasound image acquired during the sweep after the position 726 (FIGS. 7 and 8) of the ultrasound imaging probe 504. As discussed in connection with FIG. 8, in this example, the image plane 704 is fan or sector shaped. In one instance, at least an outline of one of the image plane is also provided with the 3-D visualization 1202. In the illustrated example, outlines of the first and last image planes are shown and connected to form an outline 1204 of the acquired 3-D volume for clarity and explanatory purposes. In general, a corresponding outlines is provided for one or more of the 3-D point clouds, including an outline for each of the 3-D point clouds. In one instance, the outline(s) is obtained during the segmentation and/or mask generation process. In one instance, the outline(s) visually provides the orientation of the ultrasound imaging probe 504 to the user.

In one instance, the 3-D visualization 1202 is displayed along with one or more of the 2-D ultrasound images, the segmentations, and the mask images (FIG. 9). In addition, and as described herein, a display of the 3-D visualization 1202 can be visually constructed as 2-D ultrasound image are acquired. For example, the initial displayed 3-D visualization 1202 may include a 3-D point cloud for only the first acquired 2-D ultrasound image with the displayed 3-D visualization 1202 update to include 3-D point clouds for subsequently acquired 2-D ultrasound images, as the ultrasound images become available.

A gradual build-up of the displayed 3-D visualization 1202 provides visual feedback to the sonographer. For example, the gradual build-up allows the sonographer to adjust the scan such as the speed of the sweep, the path of the sweep, imaging parameters, etc. In another example, the feedback allows the sonographer to determine early on to end the scan where it is clear that useful 3-D visualization will not be constructed. Again, since there is no 3-D reconstruction, the approach described herein mitigates the latency, time consumption, computational strain, etc. associated with 3-D reconstructions of 3-D ultrasound images from 2-D ultrasound images, and/or since the individual 3-D representations of the tissue of interest make up the displayed 3-D visualization, shortcomings associated with combining 2-D ultrasound images to create a single 3-D ultrasound image are mitigated.

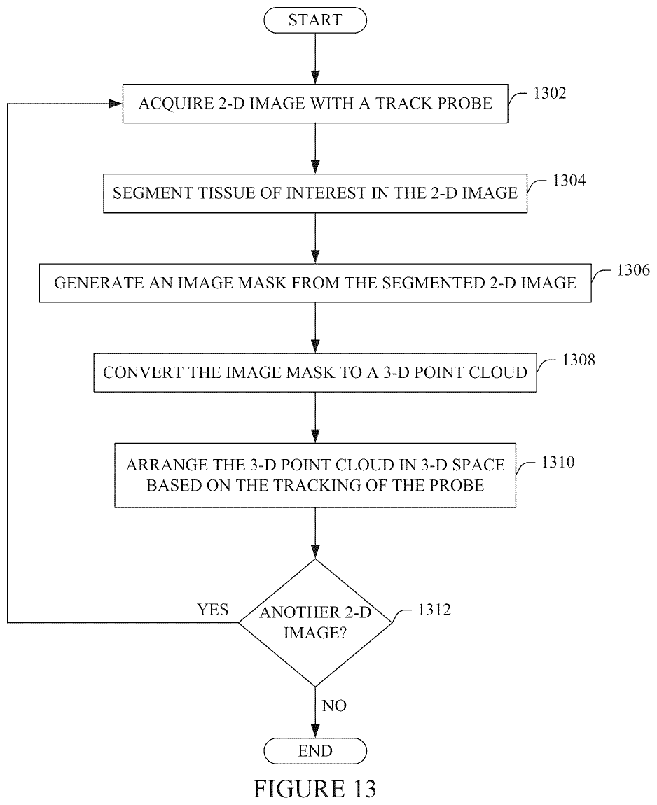

FIG. 13 illustrates a non-limiting example of a flow chart for a method of a flow for creating a 3-D visualization from 2-D ultrasound images acquired with a free hand sweep of the ultrasound imaging probe and without reconstructing the 2-D ultrasound images to create the 3-D visualization. It is to be appreciated that the ordering of the acts in the method is not limiting. As such, other orderings are contemplated herein. In addition, one or more acts may be omitted, and/or one or more additional acts may be included.

At 1302, a 2-D ultrasound image of tissue of interest is acquired while sweeping the ultrasound imaging probe, as described herein and/or otherwise. The 2-D ultrasound image can include B-mode, flow, etc. 2-D ultrasound image. In one instance, the 2-D ultrasound image is acquired during a free-hand sweep of an ultrasound imaging probe for a volume scan, where the ultrasound imaging probe includes a tracking sensor so that a position of the 2-D ultrasound image, relative to other 2-D ultrasound images acquired during the sweep, is known in 3-D space.

At 1304, the tissue of interest is segmented in the 2-D ultrasound image, as described herein and/or otherwise. As described herein, in one instance, the segmentation is fully automatic, not requiring any user input for the segmentation, while in another instance, the segmentation is semi-automatic, utilizing user input for the segmentation. In addition, the segmentation may include thresholding, clustering, AI, and/or other known and/or other approaches.

At 1306, a mask image is generated for the segmented 2-D ultrasound image, as described herein and/or otherwise. In one instance, after mask generation, the mask image will only include a representation for each region that includes the tissue of interest. In another instance, the mask image will further include an outline of the 2-D ultrasound image. For example, where the 2-D ultrasound image is a sector image, the mask image will include an outline of the sector along with the representation of the tissue of interest.

At 1308, the mask image is converted to a 3-D point cloud, as described herein and/or otherwise. For example, in one instance, each representation of the tissue of interest in the mask image is converted to a sphere with coordinates (x, y, z) by extracting the coordinates of the pixels that belong to the representation, assigning depth information to the representation to create a 3-D representation, and combining the coordinates and depth information to form the 3-D point cloud.

At 1310, the 3-D point cloud is positioned in 3-D space to create a 3-D visualization based on the ultrasound imaging probe tracking information obtained from the tracking sensor, as described herein and/or otherwise. As discussed herein, in one instance the 3-D visualization is displayed and constructed in real-time as 2-D ultrasound images are acquired. In another instance, the 3-D visualization is constructed from two or more 2-D ultrasound images before the 3-D visualization is displayed

At 1312, it is determined whether another 2-D ultrasound image will be acquired. For example, if the user still has the ultrasound imaging probe in scanning mode, acts 1302-1310 are repeated to acquire another 2-D ultrasound image during the sweep of the ultrasound imaging probe and add another 3-D point cloud to the 3-D visualization. If the user is no longer scanning with the ultrasound imaging probe, the scanning ends. The 2-D ultrasound images, the segmented images, the mask images, and/or other information can be displayed with a partial 3-D visualization and/or a final 3-D visualization.

The approach described herein generates a 3-D visualization from 2-D ultrasound images without performing a 3-D reconstruction. Rather, 3-D point clouds, each corresponding to a 2-D ultrasound image, are arranged in 3-D space based upon tracking information of the ultrasound imaging probe during the free-hand sweep of the ultrasound imaging probe during the acquisition. Since no 3-D reconstruction is performed, the approach described herein mitigates the latency, time consumption, and/or computational strain associated with 3-D reconstructions of ultrasound images. In addition, the displayed 3-D visualization can be visually constructed in near real-time, and since the individual 3-D point clouds make up the displayed 3-D visualization, shortcomings associated with combining 2-D ultrasound images to create a single 3-D ultrasound volume are mitigated.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only and are thus not intended to limit in any way the definition and/or meaning of the term “computer.” The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used in practice as some jurisdictions require them, but this should not be taken to mean that the features in the dependent claims are mutually exclusive.

What is claimed is:

1. An ultrasound imaging system, comprising:
an ultrasound imaging probe, including:
a transducer array configured to emit ultrasound pressure fields and receive echo signals during a sweep of the probe; and
a tracking sensor configured to track a spatial location and orientation of the ultrasound imaging probe during the sweep;
a beamformer configured to generate a set of 2-D ultrasound images for the sweep based on the received echo signals;
a visualization module configured to:
segment tissue of interest in each 2-D ultrasound image of the set of 2-D ultrasound images;
generate a mask image of the tissue of interest for each segmented image of the set of 2-D ultrasound images;
convert each mask image to a 3-D graphical representation of the tissue of interest; and
arrange the 3-D graphical representations in 3-D space based on corresponding tracked spatial location and orientation; and
a display monitor configured to present the 3-D graphical representations as a 3-D visualization.

2. The ultrasound imaging system of claim 1, wherein the 3-D visualization includes gaps between neighboring 3-D graphical representations.

3. The ultrasound imaging system of claim 1, wherein the 3-D graphical representation is a 3-D point cloud.

4. The ultrasound imaging system of claim 1, wherein the display monitor displays the 3-D visualization after arrangement of a 3-D graphical representation in the 3-D space.

5. The ultrasound imaging system of claim 4, wherein the display monitor updates the display of the 3-D visualization after an addition of a subsequent 3-D graphical representation in the 3-D space.

6. The ultrasound imaging system of claim 1, wherein the visualization module is configured to segment a perimeter of each 2-D ultrasound image, include the segmented perimeters in the mask image, include the segmented perimeters with the 3-D graphical representation, and display the segmented perimeters in the 3-D visualization.

7. The ultrasound imaging system of claim 1, wherein the sweep of the ultrasound imaging probe is a free-hand sweep of the ultrasound imaging probe.

8. The ultrasound imaging system of claim 1, wherein the ultrasound imaging probe is a laparoscopic ultrasound imaging probe.

9. A method, comprising:
receiving echo signals with a transducer array of an ultrasound imaging probe during a sweep of the probe;
tracking a spatial location and orientation of the ultrasound imaging probe during the sweep;
generating a set of 2-D ultrasound images for the sweep based on the received echo signals;
segmenting tissue of interest in each 2-D ultrasound image of the set of 2-D ultrasound images;
generating a mask image of the tissue of interest for each segmented image of the set of 2-D ultrasound images;
converting each mask image to a 3-D graphical representation of the tissue of interest;
arranging the 3-D graphical representations in 3-D space based on corresponding tracked spatial location and orientation; and
displaying the 3-D graphical representations as a 3-D visualization.

10. The method of claim 9, further comprising:
arranging the 3-D graphical representations in the 3-D space with gaps between neighboring 3-D graphical representations.

11. The method of claim 9, wherein the 3-D graphical representation is a 3-D point cloud.

12. The method of claim 9, further comprising:
displaying the 3-D visualization after arrangement of a 3-D graphical representation in the 3-D space.

13. The method of claim 12, further comprising:
updating the display after an addition of a subsequent 3-D graphical representation in the 3-D space.

14. The method of claim 9, further comprising:
segmenting a perimeter of each 2-D ultrasound image;
including the segmented perimeters in the mask images;
including the segmented perimeters with the 3-D graphical representation; and
displaying the segmented perimeters in the 3-D visualization.

15. A computer readable medium encoded with computer readable instructions, which, when executed by a processor, cause the processor to:
receive echo signals with a transducer array of an ultrasound imaging probe during a sweep of the probe;
track a spatial orientation of the ultrasound imaging probe during the sweep;
generate a set of 2-D ultrasound images for the sweep based on the received echo signals;
segment tissue of interest in each 2-D ultrasound image of the set of 2-D ultrasound images;
generate a mask image of the tissue of interest for each segmented image of the set of 2-D ultrasound images;
convert each mask image to a 3-D graphical representation of the tissue of interest;
arrange the 3-D graphical representations in 3-D space based on corresponding tracked spatial location and orientation; and
display the 3-D graphical representations as a 3-D visualization.

16. The computer readable medium of claim 15, wherein the instructions further cause the processor to:
arrange the 3-D graphical representations in the 3-D space with gaps between neighboring 3-D graphical representations.

17. The computer readable medium of claim 15, wherein the 3-D graphical representation is a 3-D point cloud.

18. The computer readable medium of claim 15, wherein the instructions further cause the processor to:
display the 3-D visualization after arrangement of a 3-D graphical representation in the 3-D space.

19. The computer readable medium of claim 18, wherein the instructions further cause the processor to:
update the display after an addition of a subsequent 3-D graphical representation in the 3-D space.

20. The computer readable medium of claim 15, wherein the instructions further cause the processor to:
segment a perimeter of each 2-D ultrasound image;
including the segmented perimeters in the mask images;
including the segmented perimeters with the 3-D graphical representation; and
display the segmented perimeters in the 3-D visualization.
